(19) United States
(12) Patent Application Publication
(10) Pub. No.: US 2023/0363412 A1
(43) Pub. Date: Nov. 16, 2023

(54) MICROBIAL TREATMENT

(71) Applicant: Elanco US Inc., Greenfield, IN (US)

(72) Inventor: Anna-Leigh Juliette Mary COX, Winchester (GB)

(21) Appl. No.: 17/432,725

(22) PCT Filed: Feb. 20, 2020
(86) PCT No.: PCT/US20/19002

§ 371 Date: Aug. 20, 2021

Related U.S. Application Data
(60) Provisional application No. 62/809,043, filed on Feb. 22, 2019.

(51) Int. Cl. A23K 10/18; A61K 35/742; A61K 9/00; A23K 50/75; A61P 1/14

(52) U.S. Cl. CPC A23K 10/18; A61K 35/742; A61K 9/0056; A23K 50/75; A61P 1/14

(57) ABSTRACT

The present disclosure relates to methods, compositions, and uses of a bacillus strain for providing a benefit in terms of body weight, adjusted feed conversion, and/or average daily weight gain in poultry. In particular, the present disclosure provides a method of providing Bacillus strain 747

Colonization of the gastrointestinal (GI) tract by beneficial bacteria in newly hatched poultry birds can be beneficial to the bird throughout its life (Ballou et al. (2016), Development of the chick microbiome: How early exposure influences future microbial diversity. Front. Vet. Sci. 3, 2.). Lactic acid bacteria (LAB) can play an important role in digestion and nutrient absorption, detoxification and nurturing mucosal conditions for immunological protection (Oakley and Kogut (2016), Spatial and temporal changes in the broiler chicken cecal and fecal microbiomes and correlations of bacterial taxa with cytokine gene expression. Front. Vet. Sci. 3, 11). Compositions and methods of colonizing the GI of poultry with LAB are known in the art (see, for example, US2017/0333496).

The present disclosure relates to methods, compositions, and uses of an LAB for providing a benefit in terms of body weight, adjusted feed conversion, and/or average daily weight gain in poultry. In particular, the present disclosure provides a method of providing Bacillus strain 747 (available commercially as Correlink™ ABS-747, Elanco Inc., Greenfield IN, USA).

The present disclosure provides a method of improving the average daily weight gain (ADWG) for broiler chicks via the ingestion of feed comprising Bacillus strain 747. For example, the ADWG may be improved by 3% or more, 4% or more, 5% or more.

The present disclosure provides a method of improving the average daily live gain (ADLWG) for broiler chicks via the ingestion of Bacillus strain 747. For example, the ADLWG may be improved by 1% or more, 2% or more, 3% or more.

The present disclosure provides a method of improving the feed conversion efficiency (FCE) for broiler chicks via the ingestion of Bacillus strain 747. For example, the FCE may be improved by 4% or more, 5% or more, 6% or more, 7% or more, 8% or more, 9% or more.

The present disclosure provides a method of improving the feed conversion ratio (FCR) for broiler chicks via the ingestion of Bacillus strain 747. For example, the FCR may be improved by −3% or lower, −4% or lower, −5% or lower, −6% or lower, −7% or lower, −8% or lower.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention

One or more currently preferred embodiments of the invention have been described by way of example. The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

A study was undertaken to assess the effect of Correlink™ ABS-747 Bacillus subtilis Bacillus subtilis on body weight, adjusted feed conversion and average daily live weight gain in male broilers raised under typical commercial poultry management practices for up to 42 days.

A total of 576 one-day-old male broiler chicks (Ross 308) were randomly allocated to pens on Study Day (SD) 0. This was followed by three study phases: Starter Phase I (SD 0-11); Grower Phase II (SD 11-25); Finisher Phase III (SD 25-42). During the study period, animals were issued treated feed in order to receive T1 Control (basal diet of wheat, soybean meal and barley) or T2 (basal diet plus 1.5×10⁵ CFU of Correlink™ ABS-747 Bacillus subtilis per gram of final feed.

Birds were housed in floor pens in a single environmentally controlled room. Treatment groups were assigned to pens equally using a randomised scheme. Pen numbers (1 to 96) were used during the study. Each treatment consisted of 24 replicates of 12 birds of similar weight. During each study phase, birds had ad libitum access to treatment diets and water.

Bird weights (pen weight) were measured and recorded at SD 0, 11, 25, 35 and 42. Feed issued and weighed back were recorded for each feeding phase. Bird general health, mortality and environmental temperature were recorded daily.

Performance variables of interest for each feeding period and overall included: Average live weight, average daily weight gain adjusted for mortality (ADWG), average daily live gain (ADLWG), average daily feed intake (ADFI), Gain to feed ratio (feed conversion efficiency: FCE), feed to gain ratio (feed conversion ratio: FCR).

The nutrient composition of all experimental diets and the content of Correlink™ ABS-747 Bacillus subtilis were within expected range. Birds remained in good condition throughout the study. The overall mortality (including culls) was low (2.9%). During the study period (SD 0 to 35) broilers fed diets supplemented with T2-Correlink™ ABS-747 Bacillus subtilis were 7.1% heavier and gained 5.0% more ADWG compared to T1 control (Summary Table 1). ADFI was −4.2% lower (P<0.05) in T2-Correlink™ ABS747 group compared to T1-Control group and thus resulted in 9.6% or −8.7% improved FCE and FCR respectively. Similar improvement in body weights were observed during overall study period (SD 0 to 42) where broilers fed diets supplemented with T2-Correlink™ ABS-747 Bacillus subtilis were 6.3% heavier and gained 4.1% more ADLWG compared to T1 control (Summary Table 2). However, it was worth noting that ADFI in this period was 4.1% higher (P<0.05) in T2-Correlink™ ABS747 group compared to T1-Control group and thus resulted in no significant differences (P>0.05) between treatments in FCE or FCR during the overall study period.

| | LFBW, | ADWG, | ADFI, | | |
|---|---|---|---|---|---|
| Variables | kg/bird | kg/bird/day | kg/bird/day | FCE | FCR |
| | SD 35 | SD 0 to 35 | | | |
| T1-Control | 2.1460 | 0.0620 | 0.0888 | 0.6994 | 1.4323 |
| T2-Correlink™ | 2.2990 | 0.0651 | 0.0850 | 0.7668 | 1.3072 |
| ABS747 | | | | | |
| SEM | 0.0237 | 0.0006 | 0.0008 | 0.0070 | 0.0129 |
| P-value | <.0001 | 0.0015 | 0.0013 | <.0001 | <.0001 |
| % difference | 7.1 | 5.0 | −4.2 | 9.6 | −8.7 |
| (T2 vs T1) | | | | | |

LDBW = Live Final Body Weight; ADWG = Average Daily Weight Gain adjusted for mortality; ADFI = Average Daily Feed Intake adjusted for mortality; FCE = Gain to Feed ratio adjusted for mortality; FCR = Feed to
Gain ratio adjusted for mortality;
No of replicate = 16 pens of 12 birds per treatment; SEM = Standard error of means; SD = study day
Values in a column with P ≤ 0.05 are significantly different or trending (0.05 < P ≤ 0.10).

| | LFBW, | ADWG, | ADFI, | | |
|---|---|---|---|---|---|
| Variables | kg/bird | kg/bird/day | kg/bird/day | FCE | FCR |
| | SD 42 | SD 0 to 42 | | | |
| T1-Control | 2.9469 | 0.0715 | 0.1158 | 0.6176 | 1.6195 |
| T2-Correlink™ | 3.1330 | 0.0745 | 0.1205 | 0.6185 | 1.6172 |
| ABS747 | | | | | |
| SEM | 0.0357 | 0.0007 | 0.0010 | 0.0017 | 0.0045 |
| P-value | 0.0006 | 0.0036 | 0.0021 | 0.7068 | 0.7186 |
| % difference | 6.3 | 4.2 | 4.1 | — | — |
| (T2 vs T1) | | | | | |

LDBW = Live Final Body Weight; ADWG = Average Daily Weight Gain adjusted for mortality; ADFI = Average Daily Feed Intake adjusted for mortality; FCE = Gain to Feed ratio adjusted for mortality; FCR = Feed to
Gain ratio adjusted for mortality;
No of replicate = 16 pens of 12 birds per treatment; SEM = Standard error of means; SD = study day
Values in a column with P ≤ 0.05 are significantly different or trending (0.05 < P ≤ 0.10).

A total of 576 one-day-old male broiler chicks (Ross 308) were randomly allocated to pens on Study Day (SD) 0. This was followed by three study phases: Starter Phase I (SD 0-11); Grower Phase II (SD 11-25); Finisher Phase Ill (SD 25-42). During the study period, animals were issued treated feed in order to receive T1 Control (basal diet of wheat, soybean meal and barley) or T2 (basal diet plus 1.5×10⁵ CFU of Correlink™ ABS-747 Bacillus subtilis per gram of final feed.

Birds were housed in floor pens in a single environmentally controlled room. Treatment groups were assigned to pens equally using a randomised scheme. Pen numbers (1 to 96) were used during the study. Each treatment consisted of 24 replicates of 12 birds of similar weight. During each study phase, birds had ad libitum access to treatment diets and water.

Bird weights (pen weight) were measured and recorded as SD 0, 11, 25, 35 and 42. Pen average daily weight gain, average daily feed intake and feed conversion rate and efficiency were calculated for the collection period. Bird general health, mortality and environmental temperature were recorded daily. The experimental unit was the pen. Statistical evaluations were made using ANOVA with P set at ≤1.05 for significance and at ≤1.1 for a trend towards significance. Performance variables of interest for each feeding period and overall included: bodyweight, average daily live weight gain (ADG), average daily feed intake (ADFI), feed conversion efficiency (FCE), feed conversion ratio (FCR) (adjusted and un-adjusted for mortality) and mortality by treatment.

Details of the trial product premix supplied by Church & Dwight are presented in Table 3.

TABLE 3
Details of Test and Control Premix

| | | Lot No & | | Concentration | | |
|---|---|---|---|---|---|---|
| Product | Manufacturer | Expiry | Active | CFU/g | Carrier | Storage |
| Correlink ™ | Church and | Lot No: | Bacillus | Spec: ≥3.0 × 10⁸ | 56% Limestone | Store in cool |
| ABS747 | Dwight | 06111801 | subtilis | | 43% Rice Hulls | dry location |
| Trial Product | | Exp: | strain 747 | Result: 6.0 × 10⁸ | 1% Mineral Oil | in a sealed |
| | | 11 Jun. 2019 | | | | container |

Birds were housed in floor pens in a single environmentally controlled room. Treatment groups were assigned to pens equally using a randomised scheme. Pen numbers (1 to 96) were used during the study. Each treatment consisted of 24 replicates of 12 birds of similar weight. During each study phase, birds had ad libitum access to treatment diets and water.
Animal: Broiler
Initial age: 1 day
Breed/Strain: Ross 308
Vaccinations: IB (administered by hatchery)
Sex: Male
Initial weight: Approx. 0.040 kg
Origin: P D Hook (hatcheries) Ltd
Final age: 42 days
Physical status: Healthy at trial start
Final weight: Approx. 3.023 kg

Experimental treatments are presented in Table 4.

TABLE 4
Experimental Treatments

| | | Bacillus subtilis |
|---|---|---|
| Treatment | Diet | (CFU/g feed) |
| T1 | Control (C) + probiotic-free control | 0 |
| | premix | |
| T2 | C + Bacillus subtilis ABS-747 premix | 1.5 × 10⁵ |

Nº of treatments: 2 animals/pen: 12 Replicates/treatment: 24
Animals/treatment: 288 Total Nº of replicates: 48 Total Nº of animals: 576

Upon arrival, chicks were examined by the Named Animal Care and Welfare Officer (NACWO) for signs of ill health. Any unhealthy chicks were removed and were not used for the study. Healthy chicks were randomly allocated into pens. These birds were placed into a container to obtain pen weight, and then placed into the assigned floor pen. This procedure was repeated until all pens were filled. In order to minimise differences in initial pen weights across treatments, a statistical check of mean pen weights and average/bird by treatment were conducted during bird allocation to pens. If differences were found, birds were substituted to correct the imbalance, and a pen weight collected.

Description of study phases and study design are provided in Table 5 Table 6 respectively.

TABLE 5
Study phases

| Study Phase | Study Days | Animal Weights | Feed Weighback |
|---|---|---|---|
| 1 Starter I (Mash) | 0-11 | Pen | Pen |
| 2 Grower II (Mash) | 11-25 | Pen | Pen |
| 3 Finisher III (Mash) | 25-42 | Pen | Pen |

Note:
Study Phase 3 had two weigh points: SD 35 and 42.

TABLE 6
Study Design

| | | Treatment | | | |
|---|---|---|---|---|---|
| | | Phase | | | |
| Treatment | Treatment | Duration | Nº of | Animals | Total Nº |
| Description | Dose | (days) | Pens | per pen | Animals |
| T1 | 0 CFU/g | 42 | 24 | 12 | 288 |
| (Control) | | | | | |
| T2 | 1.5 × 10⁵ | 42 | 24 | 12 | 288 |
| (Correlink™ | CFU/g | | | | |
| ABS747) | | | | | |
| Total | | | 72 | | 576 |

Trial Duration: 0 days of age to 42 days of age
Observations: Three feeding phases: Starter I (0-11 days), Grower II (11-25 days) and Finisher III (25-42 days)
Feed phase III had two weigh points: SD 25-35 and 35-42
Pen weights recorded at 0, 11, 25, 35 and 42 days on trial.
Faecal samples collected at 11, 25 and 42 days on trial
Individual pen feed weighback were measured at 11, 25, 35 and 42 days on trial.
The dead weights were recorded for all withdrawn birds.
Daily records of culling, mortality, health & environmental temperature.
Deaths and euthanasia's necropsied by veterinarian
Details of any plant malfunctions or Abnormal events (e.g. Adverse Events, severe disease outbreak, power cuts, storms, feed/water blockages, etc.).were recorded in the Study File
Calculations: Zootechnical performance parameters including average live weight, average daily feed intake, average daily live weight, weight gain, FCR and FCE (adjusted and un-adjusted for mortality) and mortality, were reported per study phase (0-11 days, 11-25, 25-42 days, 25-35 days, 35-42 days) and overall periods (0-35 and 0-42 days), were considered as primary parameters.
Nº - Number

Pre-sexed, male day old broilers (ca. 1,000 from which 576 healthy birds were selected for which study) were purchased from a commercial hatchery (P. D. Hook, Thirsk, Dalton, UK). Only healthy chicks were allocated randomly to the study pens and checked for body weight homogeneity.

Birds were managed as described below from SD 0 to 42 days of age.

| | |
|---|---|
| Housing: | From arrival until SD 42, birds were housed in House 14 of the |
| | Poultry Unit at Monogastric Science Research Centre, SRUC, Ayr. |
| | This was an environmentally controlled building with floor pens as |
| | described below. |
| Floor and Feeder | Birds were in pens measuring 1.74 m². Birds were in groups of 12 |
| Space Allocation: | birds (EU maximum stocking density at 42 d, 33 kg/m²). Each pen |
| | had nipple line and one large feed hopper. |
| Bedding: | Birds were reared on fresh wood shavings over used litter in SRUC |
| | Poultry Shed 14 (50:50 ratio). The used wood shavings from |
| | clinically healthy, untreated birds: 50%% of used litter and 50% fresh |
| | white wood shavings. Pens were deep littered, with regular checks |
| | and maintenance of litter quality. Bedding was not refreshed |
| | throughout the study period. |
| House and Heating | SRUC SOP (RD/MSRC/004) was used to set up the house. The |
| Program: | target temperatures were set to meet recommended thermal |
| | conditions for each stage of growth throughout the study period. The |
| | maximum and minimum temperatures and relative humidity within |
| | the study accommodation were recorded daily on the Temperature |
| | Record Form and Humidity Record Form as per SRUC's SOP |
| | (RD/MSRC/008). |
| Lighting Program: | Birds were given 23 hours at 1 day old, stepped down to 18 light:6 |
| | dark by 4 days of age. SOP RD/MSRC/004 was followed to set up |
| | the houses according to the species (chicken or turkey) or bird type |
| | (e.g. broiler or layer) to be housed. Light cycles were monitored by |
| | using a light meter to ensure appropriate light levels, and checking of |
| | the light set-up and alterations on a daily basis by the farm manager |
| | and Named Animal Care & Welfare Officer (referred as NACWO). |
| | Light intensity measurements were recorded on the Light Intensity |
| | form as per SRUC's SOP (RD/MSRC/008). |
| Feeding Program: | Ad libitum. Mash starter 0-11 d; mash grower 11-25 d; mash finisher |
| | 25-42 d |
| Watering Program: | Ad libitum access to water |
| Removal of Test | Birds remained with their pen group as long as they were able to get |
| Animals: | to feed and water and they did not exhibit any signs of distress or |
| | injury. |
| Euthanasia | Euthanasia was in accordance with SRUC SOP AVS/ASRC/043 |
| Procedure: | Birds <3 kg: Cervical dislocation |
| | Birds >3 kg: Electrical stunning followed by exsanguination for |
| | individual withdrawals or CO₂ gassing (ARGOSHIELD Heavy: 20% |
| | carbon dioxide, 2% oxygen, balance argon (ISO 14175-M26-ArCO- |
| | 20/2) for birds remaining at the end of the study. |
| Data Collection: | Bird weights were determined according to the schedule of events. |
| | Amount of feed issued and remaining was documented for each pen. |
| | Mortality and dead weights were documented for each pen. |

Ammonia was monitored weekly from SD 21 (and as required as decided by an experienced stockperson prior to this point) and recorded on the Ammonia Level Record Form as per SRUC SOP RD/MSRC/008. Prior to use, the room, pens and equipment were thoroughly pressure-washed and suitably disinfected according to local procedures. Re-used litter was used from clinical healthy birds untreated with any medication. The room and pens in which the birds were kept were uniquely labelled and details were retained in the Study File. A count of birds in the pen were maintained on the pen label.

Diets did not contain antibiotics, anticoccidials or growth promoters. Basal diets were calculated to be isonutritive, and met the nutrient requirements recommended for broilers. The ingredients, premix and variables for nutrient composition analyses of the diets were presented in Table 7. The diets for all phases (Starter Phase I, Grower Phase II and Finisher Phase III) were fed to the birds as a mash.

TABLE 7
Ingredients and Composition of the Experimental Diets

| | | |
|---|---|---|
| Starter | Grower | Finisher |
| Phase I | Phase II | Phase III |

Feed ingredients (%)

| | | | |
|---|---|---|---|
| Wheat Raw Ground | 50.0000 | 55.0000 | 60.0000 |
| Soya Ext Hipro | 26.0000 | 23.0000 | 19.0000 |
| Barley Raw Ground | 12.0000 | 9.9000 | 7.9500 |
| Full fat Soya Cherwell | 5.0000 | 5.0000 | 5.0000 |
| L Lysine HCL | 0.4000 | 0.3000 | 0.3000 |
| DL Methionine | 0.4000 | 0.3500 | 0.3000 |
| L Threonine | 0.1500 | 0.1500 | 0.1500 |
| Soya Oil | 2.5000 | 3.0000 | 4.0000 |
| Limestone Trucal 52 | 1.2500 | 1.2500 | 1.2500 |
| Monocalcium Phosphate | 1.5000 | 1.2500 | 1.2500 |
| Salt | 0.2500 | 0.2500 | 0.2500 |
| Sodium Bicarbonate | 0.1500 | 0.1500 | 0.1500 |
| BR. Trials PMX. STARTER 4K¹ | 0.4000 | 0.0000 | 0.0000 |
| BR. Trials PMX. GRO/FIN 4K¹ | 0.0000 | 0.4000 | 0.4000 |

Calculated chemical composition, %

| | | | |
|---|---|---|---|
| Oil EE | 4.9 | 5.4 | 6.4 |
| Protein | 21.6 | 20.4 | 18.8 |
| Fibre | 3.1 | 3.1 | 3.0 |
| ME, MJ | 12.43 | 12.69 | 13.05 |
| T lysine | 1.44 | 1.27 | 1.16 |
| Av lysine | 1.34 | 1.19 | 1.08 |
| D lys | 1.26 | 1.11 | 1.01 |
| Meth | 0.69 | 0.63 | 0.56 |
| M + C | 1.02 | 0.94 | 0.85 |
| DM + C | 0.95 | 0.88 | 0.79 |
| Threo | 0.90 | 0.85 | 0.79 |
| DTHR | 0.76 | 0.72 | 0.67 |
| Trypt | 0.25 | 0.24 | 0.22 |
| DTRY | 0.21 | 0.20 | 0.18 |
| Calcium | 0.98 | 0.93 | 0.92 |
| Phos | 0.73 | 0.66 | 0.64 |
| Avphos | 0.49 | 0.43 | 0.42 |
| Salt | 0.31 | 0.31 | 0.31 |
| Sodium | 0.18 | 0.18 | 0.18 |
| VAL | 0.80 | 0.76 | 0.69 |
| ARG | 1.32 | 1.24 | 1.12 |
| Vit A | 13.5 | 10.0 | 10.0 |
| Vit D3 | 5.0 | 5.0 | 5.0 |
| Vit E | 100.0 | 100.0 | 100.0 |

¹Provided per kilogram of diet: Vitamin A (E 672): 10,000 IU; Vitamin D3 (E 671): 2,000 IU; Vitamin E (a-tocopherol): 30.0 mg; Vitamin K3: 2.0 mg; Vitamin B1: 1.0 mg; Vitamin B2: 5.0 mg; Vitamin B6: 3.0 mg; Vitamin B12: 12.0 μg; Nicotinic acid: 40.0 mg; Calcium pantothenate: 10.0 mg; Folic acid: 1.0 mg; Biotin: 0.1 mg; Choline chloride: 400.0 mg; Cu (CuSO4•5H2O): 8.0 mg; Fe (FeCO3): 60.0 mg; I (IK): 2.0 mg; Mn (MnO): 70.0 mg; Se (Na2SeO3): 0.15 mg; Zn (ZnO): 80.0 mg; Butylated hydroxytoluene: 4.0 mg; Citric acid: 13.8 mg; Sodium citrate: 0.4 mg; Sepiolite: 0.4 g; Calcium carbonate: 2.34 g.

All experimental diets were analytically analysed for the variables as described in Table 8.

TABLE 8
Variables Assay for Nutrient Composition for T1
Starter I, Grower II and Finisher III Diets

Dry matter (%)
Crude protein
Crude fibre
Oil (A)
Ash
Gross energy
Calcium
Phosphorus
Sodium
Amino acid Profile
Amino acid profile includes: Alanine, Arginine, Aspartic Acid, Cystine, Glutamic Acid, Glycine, Histidine, Isoleucine, Leucine, Lysine, Methionine, Phenylalanine, Proline, Serine, Threonine, Tyrosine and Valine

Mash diets for each phase (Starter Phase I, Grower Phase II and Finisher Phase III) were manufactured by Target Feeds Ltd. To ensure that test and control diets had the same nutrient content, a master basal diet that contained all ingredients for each phase was manufactured. All experimental diets were prepared without antibiotics, anticoccidials, growth promotors, enzyme additives or probiotics (other than the test item) and analysed for nutritional homogeneity according to Association of Official Analytical Chemists (AOAC, 2000) or other suitable methods. The results of the proximate analyses (nutritional composition) were used to check feed quality.

From each batch (Starter I, Grower Phase II and Finisher Phase III) a control and two test diets were prepared. Trial product premixes were incorporated as described in Treatment Application (10.2). Diets containing Correlink™ ABS-747 and Correlink™ ABS-1781 were T2 and T3, respectively. The Control diet (T1) was without test product. Following preparation of gradual premixes, the basal diet and final premix was mixed in a stainless steel, horizontal, ribbon blend mixer (Alvan Blanch) for approximately 8-10 minutes. All Control diets (T1) were mixed before test diets (T2 & T3) and the mixer was flushed with wheat between test diets.

Test product (Premixes of Correlink™ ABS-747 and Correlink™ ABS-1781) and test diets were stored at ambient temperature at the test facility.

For each respective phase diet (Starter I, Grower II and Finisher III), Correlink™ premix was added to the basal diet batch to achieve a target 1.5×10⁵ CFU/g final feed. Incorporation rates, calculated from the certificate of analysis, were 250 g/1000 kg for Correlink™ ABS747 Trial Product Premix.

To insure adequate homogeneity, gradual premixes were prepared using no less than 5% inclusion at each stage. The trial product premix was initially mixed with wheat, then into series of smaller quantities of basal diet before mixing into the relevant final diets.

Bird weights (pen weights) were measured and recorded as listed below: SD 0, 11, 25, 35 and 42. All pen weights were recorded on the ‘Grouped Birds Weight Record’ (Appendix 8). On SD 0 weighing took place prior to the feeding of the test diets and on SD 11 and 25 prior to the change of phase diet and on SD 35 prior to reissue of feed.

Unscheduled bird weights included withdrawal weights of birds that died or were euthanised and were recorded on the date the bird was removed from the pen.

Test diets were fed in three phases to all treatment groups. Starter diets (Phase I) were offered from SD 0 to 11, Grower diets (Phase II) from SD 11 to 25 and Finisher diets (Phase III) from SD 25 to 42.

Each diet for phases one to three was fed to 24 replicate pens. Birds were fed on an ad libitum basis. The quantity of feed consumed from SD 0 to 11, 11 to 25, was recorded for phases one and two respectively and from 25 to 35 and 35 to 42 for phase three. Weighed feed was added to the feeders when required to ensure fresh feed was available ad libitum at all times. Feed was recorded when added. The depth of food in the lips of each feeder were kept low to avoid wastage. Feed intake was measured and recorded.

Feed weighbacks were conducted on SD 11, 25, 35 and 42 and recorded on a per pen basis and was recorded on Feed Weighbacks forms in study logbook).

24 replications per treatment group were provided at least 80% power to detect a difference of 3 points in feed conversion between control (1.653) and treatment (1.603) in broilers through 42 days assuming a standard deviation of 0.08 at alpha=0.05 using a two-sided group t-test. No adjustments were made for multiple comparisons.

Zootechnical performance parameters, including average live weight at each time point, average daily live weight gain (ADLWG), average daily weight gain (adjusted for mortality, ADWG), average daily feed intake (ADFI), FCE, and FCR (adjusted and un-adjusted for mortality), mortality and bird days alive for each study phase (Days 0-11, 11-25, 25-42) as well as 25-35, 35-42, 0-35 and 0-42), were considered the primary parameters.

Variable calculations were made relative to phases, where the phases were defined as follows: Starter phase I (0-11 days), Grower phase II (11-25 days) and Finisher phase III (25-42 days), weigh points 25-35 and 35-42 days and overall phases (0 to 35) and (0-42 days).

Performance variables were average live weight at each time point, average daily live weight gain (ADLWG), average daily weight gain (adjusted for mortality, ADWG), average daily feed intake (ADFI), FCE, and FCR (adjusted and un-adjusted for mortality), mortality and bird days alive were calculated and evaluated for each study phase (Days 0-11, 11-25 and 25-42) as well as 25-35, 35-42, 0-35 and 0-42.

Table 9 contains the formulas that were used to calculate the performance variables.

TABLE 9
Formulas

| Variable | Formula |
|---|---|
| Average live weight gain | Total weight of live birds at the end of |
| | the period/number of birds at the start |
| | of the study |
| Average daily live weight | (Total weight of live birds at the end of |
| gain (ADLWG) | the period − total weight of live birds |
| | at the start of the period)/(number of |
| | birds at start of phase * number of days |
| | in phase) |
| Average daily weight gain | (Total weight of live birds at the end of |
| adjusted for mortality | the period + weight of dead and removed |
| (ADWG) | birds − total weight of live birds at the |
| | start of the period)/((number of birds at |
| | end of phase * number of days in phase) + |
| | number of days removed birds were present |
| | within the phase) |
| Average daily feed intake | (Total feed issue − total feed |
| adjusted for mortality | weighback)/(birddays adjusted for mortality) |
| (ADFI) | |
| Average daily feed intake | (Total feed issue − total feed |
| unadjusted for mortality | weighback)/(number of birds at start |
| (un-ADFI) | of phase * number of days in phase) |
| FCR (Feed to Gain Ratio) | Un-ADFI/ADLWG |
| FCR (Feed to Gain Ratio), | ADFI/ADWG |
| adjusted for mortality | |
| FCE (Gain to Feed Ratio) | ADLWG/un-ADFI |
| FCE (Gain to Feed Ratio), | ADWG/ADFI |
| adjusted for mortality | |

All abnormal health observations are summarized in Table 10a & 10b

TABLE 10a
Listing of Abnormal Health Observations

| Observation | Study | | Treatment | System | Clinical | Cause of death |
|---|---|---|---|---|---|---|
| Date | Phase | Pen ID | No | Code | Sign Code | (Culled or Dead) |
| 7 Sep. 2018 | 1 | 52 | 1 | GEN | DEAD | Dead (Omphalitis) |
| 13 Sep. 2018 | 1 | 59 | 1 | GEN | DEAD | Dead (Septicemia) |
| 17 Sep. 2018 | 2 | 19 | 1 | GEN | DEAD | Dead (Perirenal |
| | | | | | | Hemorrhage Sudden |
| | | | | | | Death) |
| 20 Sep. 2018 | 2 | 24 | 1 | GEN | DEAD | Dead (Septicemia) |
| 23 Sep. 2018 | 2 | 50 | 1 | GEN | DEAD | Dead (Septicemia) |
| 28 Sep. 2018 | 2 | 49 | 1 | NEU | DEP | Dead (Septicemia) |
| 1 Oct. 2018 | 3 | 18 | 1 | GEN | DEAD | Dead (Septicemia) |
| 3 Oct. 2018 | 3 | 57 | 1 | GEN | STVN | Culled (Starvation) |
| 3 Oct. 2018 | 3 | 58 | 1 | INTG | SWE | Culled (Starvation) |
| 9 Oct. 2018 | 3 | 50 | 1 | CDV | HAK | Dead (Perirenal |
| | | | | | | Hemorrhage Sudden |
| | | | | | | Death) |
| 9 Oct. 2018 | 3 | 53 | 1 | CDV | HAK | Dead (Perirenal |
| | | | | | | Hemorrhage Sudden |
| | | | | | | Death) |
| 12 Oct. 2018 | 3 | 17 | 1 | GEN | DEAD | Dead (Endocarditis) |
| 19 Sep. 2018 | 2 | 11 | 2 | GEN | DEAD | Culled: Other |
| | | | | | | (Coccidial load) |
| 1 Oct. 2018 | 3 | 66 | 2 | GEN | DEAD | Dead (Other: Trauma) |
| 1 Oct. 2018 | 3 | 3 | 2 | GEN | DEAD | Dead (Septicemia) |
| 3 Oct. 2018 | 3 | 25 | 2 | MS | LAM | Culled (Lameness) |
| 14 Oct. 2018 | 3 | 11 | 2 | other | other | Dead (Other) |

T1 = control, T2 = Correlink ™ ABS747; Global Mortality % ((including culls) = 2.9% (17 birds died out of 576); Where CDV = Cardiovascular; NEU = Neurological; GEN = General; INTG = Integument (Skin and Feathers); MS = Musculoskeletal and Feet.

TABLE 10b
Summary of Abnormal Health Observations.

| | | Treatment | |
|---|---|---|---|
| | | Group* | |
| | | T1 | T2 |
| System | Clinical Sign | N | N |
| Cardiovascular | Heart attack/Sudden | 2 | 0 |
| | Death Syndrome | | |
| Gastrointestinal | Anorexia | 1 | 0 |
| General | Dead | 7 | 3 |
| | Morbid or Moribund State | 0 | 1 |
| | Starvation | 1 | 0 |
| Integument (Skin and Feathers) | Swelling | 1 | 0 |
| Musculoskeletal and Feet | Lameness | 0 | 1 |
| Neurological | Depression | 1 | 0 |

*T1 = control, T2 = Correlink ™ ABS747; N = number of observations

The summary of percent mortality analysis results is presented in Table 11. The percent mortality from day 0-35 trended to be lower in T2-Correlink™ ABS747 group when compared to T1 Control group (1.39 vs. 3.82%; P=0.0856). Whereas, no significant differences (P>0.05) were noted between treatments during SD 0-11 (starter), SD 11-25 (grower), SD 25-42 (finisher) phases and the overall study period (SD 0-42).

TABLE 11
Percent mortality during the experimental period of 42 days

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0-11 | 0.69 | 0.00 | — | 0.9707 | — |
| 11-25 | 1.39 | 0.35 | — | 0.2191 | — |
| 25-35 | 1.74 | 1.04 | — | 0.4847 | — |
| 35-42 | 0.35 | 0.35 | — | 1.0000 | — |
| 25-42 | 2.08 | 1.39 | — | 0.5294 | — |
| 0-35 | 3.82 | 1.39 | — | 0.0856 | — |
| 0-42 | 4.17 | 1.74 | — | 0.1016 | — |

No of replicate = 16 pens of 12 birds per treatment; SEM = Standard error of means; Values in a column with P ≤ 0.05 are significantly different or trending (0.05 < P ≤ 0.10).

The summary of bird days/pen analysis results is presented in Table 12. Bird days indicating liveability of birds were significantly higher in T2 Correlink™ ABS747 group during 35-42 days on trial (+2.6%; 82.75 vs. 80.63 days; P=0.0421) and 0-42 days on trial (+1.3%; 501.0 vs. 494.54 days; P=0.0386), when compared to T1 Control group. In addition, bird days trended to be higher in T2 Correlink™ ABS747 group during 11-25 days on trial (+1.2%; 167.58 vs. 165.67; P=0.0769); 25-35 days on trial (+1.7%; 118.67 vs. 116.67 days; P=0.0804); 25-42 days on trial (+2.1%; 201.42 vs. 197.29; P=0.0534); and 0-35 days on trial (+1.0%; 418.25 vs. 413.92; P=0.0544), when compared to T1 Control group.

TABLE 12
Summary of the Bird Days (total per pen)
during the experimental period of 42 days

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0-11 | 131.58 | 132.00 | 0.2405 | 0.2267 | — |
| 11-25 | 165.67 | 167.58 | 0.7489 | 0.0769 | 1.2 |
| 25-35 | 116.67 | 118.67 | 0.7910 | 0.0804 | 1.7 |
| 35-42 | 80.63 | 82.75 | 0.7186 | 0.0421 | 2.6 |
| 25-42 | 197.29 | 201.42 | 1.4710 | 0.0534 | 2.1 |
| 0-35 | 413.92 | 418.25 | 1.5523 | 0.0544 | 1.0 |
| 0-42 | 494.54 | 501.00 | 2.1442 | 0.0386 | 1.3 |

No of replicate = 16 pens of 12 birds per treatment; SEM = Standard error of means; Values in a column with P ≤ 0.05 are significantly different or trending (0.05 < P ≤ 0.10).

The results of analytical analysis of the exp. diets are provided in Table 13.

TABLE 13
Results of Analytical Analysis of Experimental Diets

| Test | | | Phase II | | Phase III | |
|---|---|---|---|---|---|---|
| (% unless | Phase I | | | Treatment | Treatment | Treatment |
| otherwise stated) | Treatment 1 | Treatment 2 | Treatment 1 | 2 | 1 | 2 |
| Oil A | 5.83 | 3.53 | 4.48 | 4.47 | 5.74 | 6.16 |
| Crude Protein | 23.30 | 23.10 | 20.80 | 20.90 | 19.50 | 19.30 |
| Crude Fibre | 3.30 | 2.80 | 3.10 | 2.90 | 2.90 | 2.50 |
| Moisture | 11.10 | 11.80 | 11.70 | 11.80 | 11.80 | 11.80 |
| Ash | 7.20 | 4.50 | 5.40 | 4.60 | 5.40 | 5.00 |
| Gross Energy | 16.37 | 16.89 | 16.80 | 16.99 | 16.82 | 17.12 |
| (MJ/Kg) | | | | | | |
| Calcium | 1.07 | 0.56 | 0.86 | 0.66 | 0.80 | 0.70 |
| Phosphorus | 0.67 | 0.51 | 0.55 | 0.49 | 0.58 | 0.51 |
| Sodium | 0.15 | 0.10 | 0.15 | 0.11 | 0.13 | 0.12 |
| Alanine | 0.81 | 0.76 | 0.73 | 0.72 | 0.69 | 0.67 |
| Arginine | 1.29 | 1.20 | 1.16 | 1.14 | 1.10 | 1.05 |
| Aspartic | 1.87 | 1.73 | 1.66 | 1.62 | 1.56 | 1.47 |
| Cystine | 0.33 | 0.32 | 0.31 | 0.30 | 0.30 | 0.29 |
| Glutamic | 4.39 | 4.31 | 4.23 | 4.16 | 4.10 | 4.05 |
| Glycine | 0.83 | 0.79 | 0.76 | 0.75 | 0.73 | 0.71 |
| Histidine | 0.49 | 0.47 | 0.46 | 0.45 | 0.43 | 0.41 |
| Iso-leucine | 0.84 | 0.80 | 0.77 | 0.75 | 0.74 | 0.70 |
| Leucine | 1.45 | 1.38 | 1.34 | 1.31 | 1.28 | 1.22 |
| Lysine | 1.36 | 1.18 | 1.13 | 1.11 | 1.07 | 0.97 |
| Methionine | 0.77 | 0.71 | 0.63 | 0.60 | 0.51 | 0.53 |
| Phenylalanine | 0.97 | 0.91 | 0.91 | 0.86 | 0.85 | 0.80 |
| Proline | 1.37 | 1.39 | 1.30 | 1.29 | 1.31 | 1.21 |
| Serine | 0.97 | 0.91 | 0.89 | 0.87 | 0.85 | 0.82 |
| Threonine | 0.91 | 0.84 | 0.83 | 0.81 | 0.80 | 0.76 |
| Tyrosine | 0.43 | 0.40 | 0.41 | 0.35 | 0.37 | 0.32 |
| Valine | 0.92 | 0.88 | 0.85 | 0.83 | 0.81 | 0.78 |

A summary of Bacillus spp. recovery analysis is presented in Table 14. Control T1 diets do not show an absence of colony forming units due to naturally occurring, heatshock-resistant, spore formers (including Bacillus spp.).

TABLE 14
Bacillus spp. Recovery

| | | | T1 | | T2 | |
|---|---|---|---|---|---|---|
| | | | Log¹⁰ | | Log¹⁰ | |
| | | | Bacillus spp. Recovery | | Bacillus spp. Recovery | |
| Diet | Point of Sample | | Results | Guarantee % | Results | Guarantee % |
| Phase | Collection | Sample Type | Log 10 | Recovery | Log 10 | Recovery |
| Starter | Manufacture | Composite | 3.42 | NA | 5.18 | 100.08 |
| | Manufacture | Homogeneity* | — | — | 5.23 | 100.95 |
| | End of diet phase | Composite | — | — | 5.08 | 103.08 |
| Grower | Manufacture | Composite | 3.53 | NA | 5.22 | 100.77 |
| | Manufacture | Homogeneity* | — | — | 5.19 | 101.33 |
| | End of diet phase | Composite | — | — | 5.24 | 101.27 |
| Finisher | Manufacture | Composite | 3.50 | NA | 5.19 | 100.30 |
| | Manufacture | Homogeneity* | — | — | 5.18 | 99.99 |
| | End of diet phase | Composite | — | — | 5.13 | 99.15 |

*Calculated as an average of 10 homogeneity samples

Body weight and weight gain analysis is summarised in Table 15 to 17.

Live final body weights were significantly higher in T2 Correlink™ ABS747 group on days 11 (+5%; 0.2979 vs. 0.2836 kg/bird; P=0.0022), 25 (+4.6%; 1.2168 vs. 1.1635 kg/bird; P=0.0123), 35 (+7.1%; 2.2990 vs. 2.1460 kg/bird; P<0.0001), and 42 (+6.3%; 3.1330 vs. 2.9469 kg/bird; P=0.0006), when compared to T1 Control group (Table 15).

TABLE 15
Live Final Body Weight, kg/bird (live birds only)

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0 | 0.0430 | 0.0432 | 0.0002 | 0.3595 | — |
| 11 | 0.2836 | 0.2979 | 0.0031 | 0.0022 | 5.0 |
| 25 | 1.1635 | 1.2168 | 0.0145 | 0.0123 | 4.6 |
| 35 | 2.1460 | 2.2990 | 0.0237 | <.0001 | 7.1 |
| 42 | 2.9469 | 3.1330 | 0.0357 | 0.0006 | 6.3 |

No of replicate = 16 pens of 12 birds per treatment; SEM = Standard error of means; SD = study day; Values in a column with P ≤ 0.05 are significantly different or trending (0.05 < P ≤ 0.10).

ADWG was significantly higher in T2 Correlink™ ABS747 during 0-11 days on trial (+5.5%; 0.0232 vs. 0.0220 kg/bird/day; P=0.0052); 25-35 (+6.2%; 0.1104 vs. 0.1040 kg/bird/day; P=0.0001); 25-42 days on trial (+4.2; 0.1153 vs. 0.1106 kg/bird/day; P=0.0036); 0-35 days on trial (+5.0%; 0.0651 vs. 0.0620 kg/bird/day; P<0.0015); and 0-42 days on trial (+4.2%; 0.0745 vs. 0.0715 kg/bird/day; P=0.0036), when compared to T1 Control group (Table 16).

TABLE 16
Average Daily Weight Gain, adjusted
for mortality (ADWG), kg/bird/day

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0-11 | 0.0220 | 0.0232 | 0.0003 | 0.0052 | 5.5 |
| 11-25 | 0.0643 | 0.0660 | 0.0010 | 0.2295 | — |
| 25-35 | 0.1040 | 0.1104 | 0.0011 | 0.0001 | 6.2 |
| 35-42 | 0.1201 | 0.1224 | 0.0017 | 0.3638 | — |
| 25-42 | 0.1106 | 0.1153 | 0.0011 | 0.0036 | 4.2 |
| 0-35 | 0.0620 | 0.0651 | 0.0006 | 0.0015 | 5.0 |
| 0-42 | 0.0715 | 0.0745 | 0.0007 | 0.0036 | 4.2 |

No of replicate = 16 pens of 12 birds per treatment; SEM = Standard error of means; SD = study day; Values in a column with P ≤ 0.05 are significantly different or trending (0.05 < P ≤ 0.10).

ADLWG was significantly higher in T2 Correlink™ ABS747 group during 0-11 days on trial (+5.9%; 0.0232 vs. 0.0219 kg/bird/day; P=0.0022); 25-35 days on trial (+8.2%; 0.1086 vs. 0.1004 kg/bird/day; P=0.0015); 25-42 days on trial (+5.5%; 0.1131 vs. 0.1072 kg/bird/day; P=0.0167); 0-35 days on trial (+7.2%; 0.0644 vs. 0.0601 kg/bird/day; P<0.0001); and 0-42 days on trial (+6.5%; 0.0736 vs. 0.0691 kg/bird/day; P=0.0006). In addition, ADLWG trended to be higher in T2 Correlink™ ABS747 group during 11-25 days on trial (+3.6%; 0.0656 vs. 0.0633 kg/bird/day; P=0.0949), when compared to T1 Control group (Table 17).

TABLE 17
Average Daily Live Gain, unadjusted
for mortality (ADLWG), kg/bird/day

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0-11 | 0.0219 | 0.0232 | 0.0003 | 0.0022 | 5.9 |
| 11-25 | 0.0633 | 0.0656 | 0.0010 | 0.0949 | 3.6 |
| 25-35 | 0.1004 | 0.1086 | 0.0017 | 0.0015 | 8.2 |
| 35-42 | 0.1191 | 0.1207 | 0.0025 | 0.6480 | — |
| 25-42 | 0.1072 | 0.1131 | 0.0017 | 0.0167 | 5.5 |
| 0-35 | 0.0601 | 0.0644 | 0.0007 | <.0001 | 7.2 |
| 0-42 | 0.0691 | 0.0736 | 0.0008 | 0.0006 | 6.5 |

No of replicate = 16 pens of 12 birds per treatment; SEM = Standard error of means; SD = study day; Values in a column with P ≤ 0.05 are significantly different or trending (0.05 < P ≤ 0.10).

Feed Intake adjusted and unadjusted for mortality summary statistics analysis are presented in Table 18 to 19 respectively.

Table 18 shows that the ADFI adjusted for mortality was significantly higher in T2 Correlink™ ABS747 group during 35-42 days on trial (+18.1%; 0.3004 vs. 0.2544 kg/bird/day; P<0.0001); 25-42 days on trial (+4.2%; 0.2002 vs. 0.1921 kg/bird/day; P=0.0018); and 0-42 days on trial (+4.1%; 0.1205 vs. 0.1158 kg/bird/day; P=0.0021). However, ADFI was significantly lower in T2 Correlink™ ABS747 group during 25-35 days on trial (−12.5%; 0.1303 vs. 0.1489 kg/bird/day; P<0.0001) and 0-35 days on trial (−4.2%; 0.0850 vs. 0.0888 kg/bird/day; P=0.0013).

TABLE 18
Average Daily Feed Intake (ADFI),
adjusted for mortality, kg/bird/day

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0-11 | 0.0273 | 0.0276 | 0.0003 | 0.3664 | — |
| 11-25 | 0.0953 | 0.0980 | 0.0011 | 0.1040 | — |
| 25-35 | 0.1489 | 0.1303 | 0.0023 | <.0001 | −12.5 |
| 35-42 | 0.2544 | 0.3004 | 0.0053 | <.0001 | 18.1 |
| 25-42 | 0.1921 | 0.2002 | 0.0017 | 0.0018 | 4.2 |
| 0-35 | 0.0888 | 0.0850 | 0.0008 | 0.0013 | −4.2 |
| 0-42 | 0.1158 | 0.1205 | 0.0010 | 0.0021 | 4.1 |

No of replicate = 16 pens of 12 birds per treatment; SEM = Standard error of means; SD = study day; Values in a column with P ≤ 0.05 are significantly different or trending (0.05 < P ≤ 0.10).

Table 19 shows that ADFI unadjusted for mortality was significantly higher in T2 Correlink™ ABS747 group during 35-42 days on trial (+18.2%; 0.3000 vs. 0.2539 kg/bird/day; P<0.0001); 25-42 days on trial (+4.5%; 0.1983 vs. 0.1897 kg/bird/day; P=0.0034); and 0-42 days on trial (+5.5%; 0.1198 vs. 0.1136 kg/bird/day; P=0.0001); and trended to be higher during 11-25 days on trial (+3.3%; 0.0977 vs. 0.0946 kg/bird/day; P=0.0537). However, ADFI (unadjusted for mortality) was significantly lower in T2 Correlink™ ABS747 group during 25-35 days on trial (−12.6%; 0.1293 vs. 0.1479 kg/bird/day; P<0.0001) and 0-35 days on trial (−3.2%; 0.0846 vs. 0.0874 kg/bird/day; P=0.0082), when compared to T1 Control group.

TABLE 19
Average Daily Feed Intake (un-ADFI),
unadjusted for mortality, kg/bird/day

| | Treatment Group | | | | |
|---|---|---|---|---|---|
| | | T2 - | | | |
| Study | T1 - | Correlink ™ | | P-value | % difference |
| Day | Control | ABS747 | SEM | (T1 vs. T2) | (T2 vs. T1) |
| 0-11 | 0.0272 | 0.0276 | 0.0003 | 0.2724 | — |
| 11-25 | 0.0946 | 0.0977 | 0.0011 | 0.0537 | 3.3 |
| 25-35 | 0.1479 | 0.1293 | 0.0023 | <.0001 | −12.6 |
| 35-42 | 0.2539 | 0.3000 | 0.0053 | <.0001 | 18.2 |
| 25-42 | 0.1897 | 0.1983 | 0.0020 | 0.0034 | 4.5 |
| 0-35 | 0.0874 | 0.0846 | 0.0007 | 0.0082 | −3.2 |
| 0-42 | 0.1136 | 0.1198 | 0.0011 | 0.0001 | 5.5 |

No of replicate = 16 pens of 12 birds per treatment; SEM = Standard error of means; SD = study day; Values in a column with P ≤ 0.05 are significantly different or trending (0.05 < P ≤ 0.10).

Feed Conversion data is summarised in Table 20 to 23.

Table 20 shows that FCE data after adjusted for mortality was significantly higher in T2 Correlink™ ABS747 group during 0-11 days on trial (+4.0%; 0.8394 vs. 0.8074; P=0.0190); 25-35 days on trial (+21.8%; 0.8544 vs. 0.7015; P<0.0001) and 0-35 days on trial (+9.6%; 0.7668 vs. 0.6994; P<0.0001); and significantly lower during 35-42 days on trial (−13.6%; 0.4101 vs. 0.4744; P<0.0001), when compared to T1 Control group.

TABLE 20
Gain to Feed ratio, FCE (adjusted for mortality) Treatment Group

| | Treatment Group | | | | |
|---|---|---|---|---|---|
| | | T2 - | | | |
| Study | T1 - | Correlink ™ | | P-value | % difference |
| Day | Control | ABS747 | SEM | (T1 vs. T2) | (T2 vs. T1) |
| 0-11 | 0.8074 | 0.8394 | 0.0093 | 0.0190 | 4.0 |
| 11-25 | 0.6744 | 0.6728 | 0.0032 | 0.7331 | — |
| 25-35 | 0.7015 | 0.8544 | 0.0157 | <.0001 | 21.8 |
| 35-42 | 0.4744 | 0.4101 | 0.0077 | <.0001 | −13.6 |
| 25-42 | 0.5757 | 0.5763 | 0.0023 | 0.8491 | — |
| 0-35 | 0.6994 | 0.7668 | 0.0070 | <.0001 | 9.6 |
| 0-42 | 0.6176 | 0.6185 | 0.0017 | 0.7068 | — |

Table 21 shows that FCE data unadjusted for mortality was significantly higher in T2 Correlink™ ABS747 group during 0-11 days on trial (+4.4%; 0.8394 vs. 0.8040; P=0.0110); 25-35 days on trial (+23.9%; 0.8462 vs. 0.6831; P<0.0001); 0-35 days on trial (+10.8%; 0.7626 vs. 0.6880; P<0.0001); and significantly lower during 35-42 days on trial (−13.9%; 0.4051 vs. 0.4706; P<0.0001), when compared to T1 Control.

TABLE 21
Gain to Feed ratio, FCE (unadjusted for mortality)

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0-11 | 0.8040 | 0.8394 | 0.0094 | 0.0110 | 4.4 |
| 11-25 | 0.6687 | 0.6713 | 0.0040 | 0.6423 | — |
| 25-35 | 0.6831 | 0.8462 | 0.0181 | <.0001 | 23.9 |
| 35-42 | 0.4706 | 0.4051 | 0.0097 | <.0001 | −13.9 |
| 25-42 | 0.5647 | 0.5700 | 0.0051 | 0.4693 | — |
| 0-35 | 0.6880 | 0.7626 | 0.0082 | <.0001 | 10.8 |
| 0-42 | 0.6086 | 0.6139 | 0.0034 | 0.2847 | — |

No of replicate = 16 pens of 12 birds per treatment; SEM = Standard error of means; SD = study day; Values in a column with P ≤ 0.05 are significantly different or trending (0.05 < P ≤ 0.10).

Table 22 shows that FCR data after adjusted for mortality was significantly lower in T2 Correlink™ ABS747 group during 0-11 days on trial (−3.6%; 1.1959 vs. 1.2402; P=0.0153), 25-35 days on trial (−17.5%; 1.1842 vs. 1.4349; P<0.0001) and 0-35 days on trial (−8.7%; 1.3072 vs. 1.4323; P<0.0001); and significantly higher during 35-42 days on trial (+16.5%; 2.4648 vs. 2.1160; P<0.0001).

TABLE 22
Feed to Gain ratio, FCR (adjusted for mortality)

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0-11 | 1.2402 | 1.1959 | 0.0124 | 0.0153 | −3.6 |
| 11-25 | 1.4836 | 1.4871 | 0.0071 | 0.7345 | — |
| 25-35 | 1.4349 | 1.1842 | 0.0258 | <.0001 | −17.5 |
| 35-42 | 2.1160 | 2.4648 | 0.0420 | <.0001 | 16.5 |
| 25-42 | 1.7376 | 1.7358 | 0.0071 | 0.8590 | — |
| 0-35 | 1.4323 | 1.3072 | 0.0129 | <.0001 | −8.7 |
| 0-42 | 1.6195 | 1.6172 | 0.0045 | 0.7186 | — |

No of replicate = 16 pens of 12 birds per treatment; SEM = Standard error of means; SD = study day; Values in a column with P ≤ 0.05 are significantly different or trending (0.05 < P ≤ 0.10).

FCR data (unadjusted for mortality) was significantly lower in T2 Correlink™ ABS747 group during 0-11 days on trial (−4.0%; 1.1959 vs 1.2457; P=0.0080); 25-35 days on trial (−19.3%; 1.1975 vs. 14841; P<0.0001); 0-35 days on trial (−9.9%; 1.3147 vs. 1.4584; P<0.0001); and significantly higher during 35-42 days on trial (+18.4%; 2.5310 vs. 2.1375; P=0.0005), when compared to T1 control group (Table 23).

TABLE 23
Feed to Gain ratio, FCR (unadjusted for mortality)

| | Treatment Group | | | | |
|---|---|---|---|---|---|
| | | T2 - | | | % |
| Study | T1 - | Correlink ™ | | P-value | difference |
| Day | Control | ABS747 | SEM | (T1 vs. T2) | vs. T1 |
| 0-11 | 1.2457 | 1.1959 | 0.0127 | 0.0080 | −4.0 |
| 11-25 | 1.4971 | 1.4906 | 0.0090 | 0.6088 | — |
| 25-35 | 1.4841 | 1.1975 | 0.0335 | <.0001 | −19.3 |
| 35-42 | 2.1375 | 2.5310 | 0.0743 | 0.0005 | 18.4 |
| 25-42 | 1.7748 | 1.7576 | 0.0171 | 0.4796 | — |
| 0-35 | 1.4584 | 1.3147 | 0.0161 | <.0001 | −9.9 |
| 0-42 | 1.6442 | 1.6302 | 0.0096 | 0.3076 | — |

No of replicate = 16 pens of 12 birds per treatment; SEM = Standard error of means; SD = study day; Values in a column with P ≤ 0.05 are significantly different or trending (0.05 < P ≤ 0.10).

General health was good and no adverse effects were observed at the supplemented dose of Correlink™ ABS747.

In comparison with T1 Control, birds fed 1.5×10⁵ CFU Correlink™ ABS747/g feed significantly improved the final body weight and the average weight gain for the overall study periods (P<0.05).

Feed intake was significantly lower during SD 25-35 day and higher during SD 35-42 day in Correlink™ ABS747 group compared to T1 control group.

FCR and FCE data suggests that inclusion of 1.5×10⁵ Correlink™ ABS747/g feed is significantly more efficient during SD0-35 day period compared to T1 Control.

The overall mortality (2.9%) was within the normal range and that it was not associated with the test product (Correlink™ ABS747).

1.-16. (canceled)

17. A method of feeding broiler chickens, comprising feeding said broiler chickens a sufficient amount of Bacillus strain 747 to provide at least one of:
an average daily weight gain (ADWG) of at least 3%;
an average daily live gain (ADLWG) of at least 1%;
a feed conversion efficiency (FCE) of at least 6%; and
feed conversion ratio (FCR) of at least −6%.

18. The method of claim 17, wherein the ADWG comprises at least 5%.

19. The method of claim 17, wherein the ADLWG comprises at least 3%.

20. The method of claim 17, wherein the FCE comprises at least 9%.

21. The method of claim 17, wherein the FCR comprises at least −8%.

22. A method, comprising feeding broiler chickens a sufficient amount of Bacillus strain 747 to provide an improved performance variable, wherein the improved performance variable comprises at least one of:
an average daily weight gain (ADWG);
an average daily live gain (ADLWG);
a feed conversion efficiency (FCE); and
feed conversion ratio (FCR).

23. The method of claim 22, wherein the ADWG comprises at least 3%.

24. The method of claim 22, wherein the ADWG comprises at least 5%.

25. The method of claim 22, wherein the ADLWG comprises at least 1%.

26. The method of claim 22, wherein the ADLWG comprises at least 3%.

27. The method of claim 22, wherein the FCE comprises at least 6%.

28. The method of claim 22, wherein the FCE comprises at least 9%.

29. The method of claim 22, wherein the FCR comprises at least −6%.

30. The method of claim 22, wherein the FCR comprises at least −8%.
